(19) United States
(12) Patent Application Publication
(10) Pub. No.: US 2023/0181005 A1
(43) Pub. Date: Jun. 15, 2023

(54) HANDHELD SURGICAL DEVICE AND SHAFT FOR A HANDHELD SURGICAL DEVICE

(71) Applicant: OLYMPUS WINTER & IBE GMBH, Hamburg (DE)

(72) Inventors: Bastian SCHRÖDER, Hamburg (DE); Andreas RÜHS, Ahrensburg (DE); Kevin Alexander SCHULZ, Wilstedt (DE); Irina SCHMUCK, Heide (DE)

(73) Assignee: OLYMPUS WINTER & IBE GMBH, Hamburg (DE)

(21) Appl. No.: 18/077,874

(22) Filed: Dec. 8, 2022

Related U.S. Application Data
(60) Provisional application No. 63/288,866, filed on Dec. 13, 2021.

(51) Int. Cl. A61B 1/00; A61B 1/018

(52) U.S. Cl. CPC A61B 1/0008; A61B 1/018; A61B 1/307

(57) ABSTRACT

A shaft and a handheld surgical device have an optimized shape for accommodating an instrument. This is achieved in that a peripheral border of a distal opening of a shaft for a handheld surgical device has a thickening facing outward at least in sections. Cut injuries can be avoided by this thickening due to the rounded borders of the distal opening.

The invention relates to a shaft for a handheld surgical device according to the preamble of claim 1. Furthermore, the invention relates to a handheld surgical device as claimed in claim 10.

Handheld surgical devices, for example, endoscopes, resectoscopes, cystoscopes, and the like are essentially constructed from a device body or main body and a tubular shaft. The tubular shaft can be, for example, an external shaft. Various further tubular shafts, for example, internal shafts, can be mounted in the interior of the external shaft in parallel to the longitudinal direction of the external shaft. The internal shafts can contain various instruments for medical treatment, for example, an optical unit or an electrode carrier in the case of a resectoscope. It is provided for most instruments here that they are movable in the external shaft in parallel to the longitudinal direction thereof. In addition to the optical unit, still further instruments can be inserted into the shaft. The space which is not occupied by the optical unit in the shaft is referred to as the operating channel. The further instruments may be inserted into the shaft in this operating channel. In addition to the external circumference of the shaft, the size or the diameter of the operating channel represents an important parameter of the handheld surgical device.

Handheld surgical devices of the type described here are primarily used for endoscopic applications in the human body. For this purpose, the tubular shaft of a handheld surgical device is inserted in each case into an opening of the human body. The distal end of the external shaft and possibly also the distal ends of internal shafts come into direct contact with the human tissue at this time. This contact in principle has a risk of injury for the human tissue. The risk of injury originates in particular from the edge of the distal end of the tubular shaft. For example, cystoscopes are used for diagnostic or therapeutic methods in the lower urinary tract. The urethra of the patient cannot be expanded arbitrarily here and thus restricts the circumference of the shaft of the cystoscope which can be inserted into the patient. So as not to injure the patient, the shaft is not supposed to be larger than is anatomically possible. If a shaft having a larger circumference cannot be used, this also means that certain instruments possibly cannot be used for the treatment of the patient, since the operating channel would be too small. Efforts have therefore been made to use a shaft, the operating channel of which has an optimum value in relation to the external circumference of the shaft. For optimization of the ratio of external circumference of the shaft and diameter of the operating channel, first a profile shape of the shaft is to be selected which forms the smallest possible dead spaces within the shaft. In addition, all manufacturing tolerances are to be reduced in order to ensure a minimum passage. A further possibility for reducing the dimension of the shaft is to reduce the wall thickness of the shaft and to avoid constrictions inside the shaft. Shafts having an external circumference reduced in this way cannot have sharp edges or borders at the ends, in particular at the distal end, however. For this purpose, it is known that borders of the shaft have a thickening to avoid the risk of traumatization of the patient. However, such thickenings have the result that the internal circumference of the shaft is reduced. The volume of the operating channel for accommodating instruments is thus also reduced.

The present invention is based on the object of providing a shaft and a handheld surgical device which has an optimized shape for accommodating an instrument.

An achievement of this object is described by the measures of claim 1. It is accordingly provided that a peripheral border of a distal opening of a shaft for a handheld surgical device has a thickening facing outward at least in sections, in particular a material thickening. Cut injuries to the patient can be avoided by this thickening due to the rounded borders of the distal opening. By displacing the thickening outward, the interior or the operating channel of the shaft is released for the insertion of an instrument. The size of the operating channel is thus optimized or maximized. The insertion of the handheld device or shaft into the human body may also be improved by the claimed enlargement in sections of the outer periphery of the shaft. It has proven to be advantageous in particular in relation to an instrument, the external diameter of which is enlarged over the entire length of the shaft.

The invention provides in particular that the entire peripheral border of the distal opening has the thickening facing outward. A traumatization of the patient can be quasi-precluded by this continuous thickening of the border. In addition, an increased stability of the shaft, in particular in the distal region, can be achieved by the thickening.

One particularly preferred exemplary embodiment of the invention provides that the distal opening is formed beveled. Due to this beveled profile or due to this beveled formation of the opening, the external circumference of the shaft is reduced toward the distal end. This reduction of the external circumference can take place continuously or step-by-step depending on the form of the bevel. An essential feature of the invention here is that the external circumference or diameter of the shaft decreases in spite of the thickening of the peripheral border of the beveled distal end. The external diameter of the shaft is only increased due to the thickening in relation to the rest of the shaft in the region of the shaft located farthest proximally, in which the bevel begins. Furthermore, it can be provided that the external circumference of the shaft is enlarged by the thickening of the border only over a length of the shaft of 0.5 mm to 5 mm, in particular of 1 mm. In addition, it is conceivable that the external circumference of the shaft is only enlarged by the thickening of the border over a length of the shaft of 0.1% to 1.0%, in particular 0.5%. These values have proven to be particularly advantageous for their application.

The invention preferably provides that the border of the distal opening has two beveled regions and one horizontal region, which is aligned at least nearly in parallel to a longitudinal axis of the shaft. Such a stepped shape of the distal opening is particularly well suitable for the insertion of the device into the human body and for the use of the optical unit in conjunction with a further device, which is to be guided through the operating channel of the shaft.

It is conceivable according to the invention that the thickness of the thickening of the opening changes continuously along the tube. This continuous change of the dimensioning of the thickening may be implemented by an optimized shape of the opening for the insertion into the body and the use of further instruments inside the shaft. In addition, the stability of the shaft may be maximized by an adapted thickening of the opening, and with reduction of the material at the same time.

One particularly advantageous exemplary embodiment of the invention can provide that the thickening has a round, preferably a circular cross section. Such shapes are optimally adapted to the anatomical conditions. However, it can also prove to be advantageous if the thickening has a different shape, for example, to increase the stability or save material.

A handheld surgical device for achieving the object stated at the outset is described by the features of claim 10. It is accordingly provided that a handheld surgical device, in particular a cystoscope, a resectoscope, or the like has a shaft as claimed in at least one of claims 1-9.

A preferred exemplary embodiment of the invention will be described in more detail hereinafter on the basis of the drawings. In the figures:

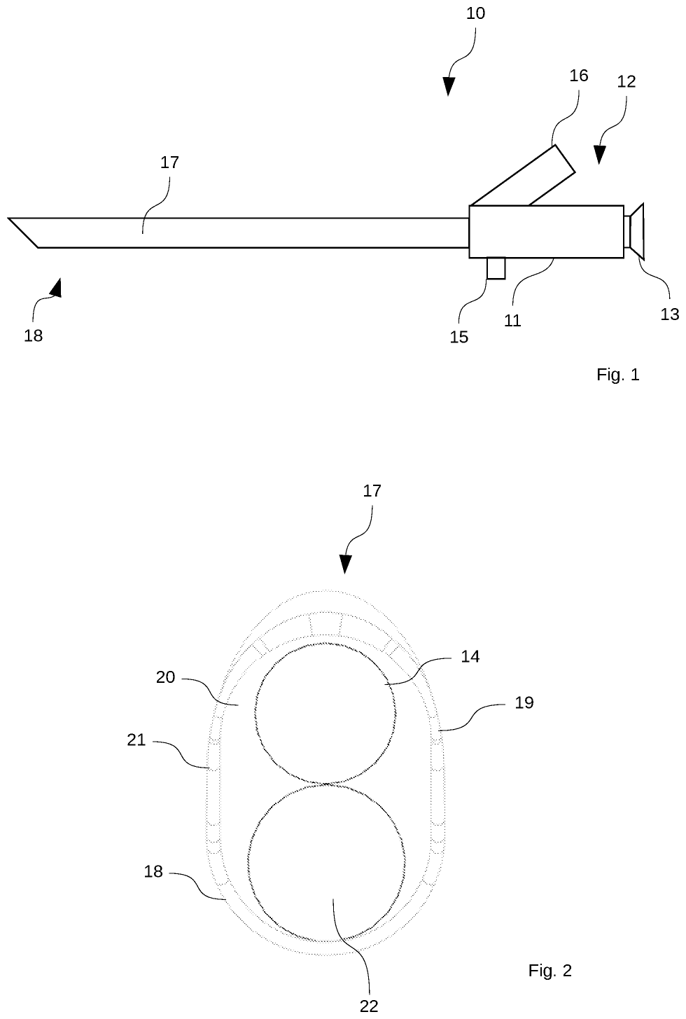

FIG. 1 shows a schematic illustration of a handheld surgical device,

FIG. 2 shows a schematic view of a distal end of the handheld surgical device,

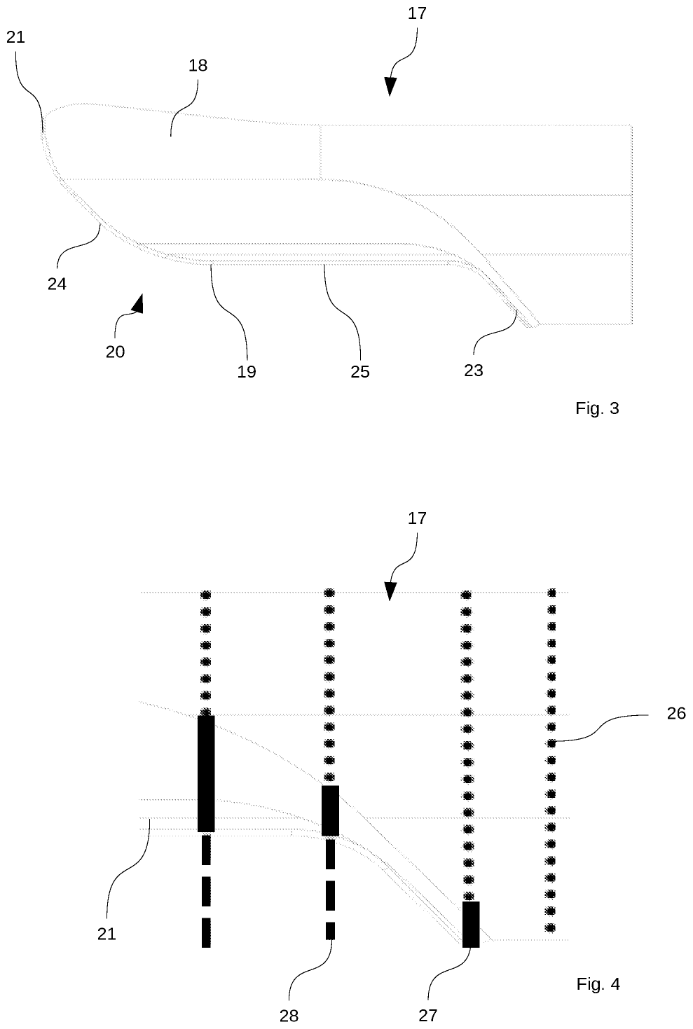

FIG. 3 shows a schematic illustration of a distal end section of a tubular shaft, and

FIG. 4 shows a schematic illustration of a region of the distal end section.

A handheld surgical device 10 is illustrated very schematically and in symbolic form for better clarity in FIG. 1. This handheld surgical device 10 has a device body 11, which can also be formed as a handle unit. Furthermore, an eyepiece 13 for an optical unit 14 is arranged at a proximal end 12 of the device body 11. In addition, the device body 11, which is shown solely schematically here, has two fittings 15, 16. These fittings 15, 16 can be used to supply the handheld surgical device 10 with electrical energy, light, a fluid, or as a port for further instruments.

A tubular shaft 17 is arranged at an end opposite to the proximal end 12 of the device body 11. This shaft 17 is formed oblong and is used to be inserted with a distal end 18 into the patient. This shaft 17 is also shown very schematically in FIG. 1 and is only used for better comprehension of the invention described here. It is to be expressly noted here that such a shaft 17 can also be coupled to other surgical devices. The beveled formation of the distal end 18 of the shaft 17 is thus also to be considered very schematic. This is because it is certainly provided that the shape of the bevel can deviate strongly from that shown in FIG. 1.

The invention provides that a peripheral border 19 of a distal opening 20 of the shaft 17 has a thickening 21 at least in sections. This thickening 21 of the peripheral border 19 is shown in FIG. 2. FIG. 2 shows a view of the distal opening 20 of the shaft 17. It can be seen in the exemplary embodiment shown here that the thickening 21 extends over the entire peripheral border 19. The external circumference or the diameter of the shaft 17 is enlarged in regions by this thickening 21. Since this thickening 21 extends outward, the internal circumference remains uninfluenced by this thickening 21, i.e., the full internal volume of the shaft 17 is available to insert or feed through instruments therein.

It is apparent from FIG. 2 that in the exemplary embodiment illustrated here, a very schematic optical unit 14, which can be formed as an optical fiber or rod lens system, is arranged inside the shaft 17. The space around the optical unit 14 or below the optical unit 14 is referred to as the operating space. Further instruments may be inserted through the shaft 17 into the interior of the patient to be treated through this operating space. In the exemplary embodiment illustrated here, this operating space is indicated as a circle 22. Due to the extension of the thickening 21 out of the interior of the shaft 17 outward, the greatest possible volume is available to the optical unit 14 and the operating space.

As already stated above, this thickening 21 of the peripheral border 19 is absolutely necessary so as not to traumatize the patient with the sharp edges of the distal end 18 of the shaft 17. The thickening 21 can have a circular, oval, or similar cross section here having a diameter of a few tenths of millimeters.

The distal end 18 of the shaft 17 is shown from the side in FIG. 3. It is apparent from the illustration that the distal opening 20 is beveled. The shape of this bevel is made beak-like here. While the opening 20 has two beveled regions 23, 24, a horizontal section extends between these two regions 23, 24. It has been shown that this shape of the distal opening 20 is particularly well suitable to insert the shaft 17 into the patient in a manner gentle to the body. In particular, traumatization or injury can be avoided by the thickening 21 of the peripheral border 19. At the same time, instruments, for example, an optical unit 14, may be guided efficiently through the shaft 17 due to the displacement of the thickening 21 outward.

The external circumference of the shaft 17 is only increased by the thickening 21 on a very short section by the shape illustrated here of the distal opening 20 of the shaft 17. This is illustrated in FIG. 4. FIG. 4 shows a detail of the distal opening 20 according to FIG. 3. The dotted line 26 represents the unchanged region of the shaft 17, which corresponds to the diameter of the shaft 17. The region of the shaft 17 is identified by the solid stroke 27, the external circumference of which is increased by the thickening 21 in comparison to the remaining shaft 17 (dotted line 26). The dot-dash line 28 identifies the region of the shaft 17 at which the external circumference or the diameter is reduced by the shape of the distal opening 20. Because the thickening 21 follows the beveled distal opening 20, the external circumference of the largest cross section is only slightly increased. Only approximately 1 mm before the beginning of the distal opening 20, the external circumference is slightly increased. With the beveling of the distal opening 20, the external circumference of the shaft 17 decreases in spite of the thickening 21. It has been shown that the shaft 17 may be inserted with a very locally increased external circumference better into the patient than a shaft which has a correspondingly enlarged external circumference over the entire length of the shaft.

### LIST OF REFERENCE NUMERALS

10 handheld surgical device
11 device body
12 proximal end
13 eyepiece
14 optical unit
15 fitting
16 fitting
17 shaft
18 distal end
19 peripheral border
20 distal opening
21 thickening
22 circle
23 oblique region
24 oblique region
25 horizontal section
26 dotted line
27 solid stroke
28 dot-dash line

1. A shaft for a handheld surgical device for accommodating an optical unit and at least one further instrument, wherein a proximal end of the tubular shaft can be coupled to a device body and/or a handle unit and wherein the optical unit and the instrument are positionable with their distal ends inside a distal opening of the shaft, wherein a peripheral border of the distal opening of the shaft has a thickening facing outward at least in sections.

2. The shaft for a handheld surgical device as claimed in claim 1, wherein the entire peripheral border of the distal opening has the thickening facing outward.

3. The shaft for a handheld surgical device as claimed in claim 1, wherein the distal opening is formed beveled.

4. The shaft for a handheld surgical device as claimed in claim 1, wherein the border of the distal opening has two beveled regions, and a horizontal section, which is aligned at least nearly in parallel to a longitudinal axis of the shaft.

5. The shaft for a handheld surgical device as claimed in claim 1, wherein the thickness of the thickening of the opening changes continuously along the shaft.

6. The shaft for a handheld surgical device as claimed in claim 1, wherein an external circumference of the shaft is only enlarged in regions by the thickening of the border.

7. The shaft for a handheld surgical device as claimed in claim 6, wherein the external circumference of the shaft is enlarged by the thickening of the border only over a length of the shaft of 0.5 mm to 5 mm.

8. The shaft for a handheld surgical device as claimed in claim 6, wherein the external circumference of the shaft is enlarged by the thickening of the border only over a length of the shaft of 0.1% to 1.0%.

9. The shaft for a handheld surgical device as claimed in claim 1, wherein the thickening has a round cross section.

10. A handheld surgical device having a shaft as claimed in claim 1.
